(19) United States
(12) Patent Application Publication
(10) Pub. No.: US 2017/0016831 A1
(43) Pub. Date: Jan. 19, 2017

(54) SYSTEMS AND METHODS FOR DETERMINING CONCENTRATION OF A COMPONENT IN A FLUID SAMPLE

(71) Applicants: Fan WU, Liuzhou, Guangxi (CN); EMPIRE TECHNOLOGY DEVELOPMENT LLC, Wilmington, DE (US)

(72) Inventor: Fan WU, Liuzhou (CN)

(73) Assignee: EMPIRE TECHNOLOGY DEVELOPMENT LLC, Wilimington, DE (US)

(21) Appl. No.: 15/122,001

(22) PCT Filed: Feb. 28, 2014
(86) PCT No.: PCT/CN2014/072663

§ 371 Date: Aug. 26, 2016

(51) Int. Cl. G01N 21/82; G01N 33/53; G01N 33/536; G01N 15/06; G01N 21/51

(52) U.S. Cl. CPC G01N 21/82; G01N 15/06; G01N 21/51; G01N 33/536; G01N 33/5304; G01N 2015/0053

(57) ABSTRACT

Systems and methods for determining concentration of a component of a fluid sample are described. The system includes a reaction vessel (30), a light source (60), a detection tube (70), and a mobile terminal (80), wherein the system is configured to determine the concentration of the component in the fluid sample based on characteristics of scattered light produced from the interaction of the component with a detection compound.

### FIELD

Disclosed herein are systems and methods for determining a concentration of a component in a fluid sample.

### BACKGROUND

Many analyses and diagnostics involve determining concentrations of components in fluid samples. For example, evaluation of the presence of a pollutant in a water source involves determining the concentration of the pollutant. Additionally, detection of, for example, a protein that is indicative of a particular disease, requires a specific determination of the concentration of that protein. Traditional analyzers may be used to determine the concentration of a target component. However, such analyzers employ highly specialized instrumentation which may limit their overall applicability and widespread use.

Current systems and techniques for detecting concentrations of components of interest in fluid samples have various deficiencies that limit their overall applicability and widespread use. For example, where the detection of a component is required to determine a medical condition (for example, a protein or antigen of interest), the detection has to be performed in a hospital or a medical care facility, largely due to the use of specialized instruments. This requirement can result in possible delays of diagnosis and treatment of some acute diseases, especially for populations without ready access to advanced equipment. Moreover, such highly specialized equipment requires trained personnel, and in some cases, medical professionals. Often times, there can be a limited number of qualified operators, which can limit the widespread use of such systems and methods. There may also be a large risk of errors in cases where individuals with inadequate training operate the equipment. Many of these traditional systems with specialized equipment can be expensive, and typically cannot be easily relocated (for example, they occupy a permanent space in a lab). Thus, there is a need for systems that can determine concentrations of components in fluid samples, such as systems that can be operational in mobile situations (such as at home, in rural locations, and in small clinics, and the like).

### SUMMARY

Some embodiments disclosed herein relate to systems for determining the concentration of a component in a fluid sample. In one embodiment, a system for determining the concentration of a component in a fluid sample may include a reaction vessel, a light source, and a detection tube. The reaction vessel may be configured to contain the fluid sample and a first reagent, and may be configured to receive a second reagent with a detection compound capable of interacting with the component in the fluid sample. The reaction vessel may include at least a first window. The light source may be configured to emit light into the reaction vessel, such that the detection compound and the component, when interacted and exposed to the light entering the reaction vessel, produces scattered light. The first window of the reaction vessel may be configured to allow the scattered light to exit the reaction vessel. The detection tube may include a first end and a second end, and may be configured to transmit the scattered light from the first end to the second end. The first end of the detection tube may be configured to engage the first window of the reaction vessel. The second end of the detection tube may be configured to reversibly engage a mobile terminal. The mobile terminal may be configured to determine the concentration of the component of interest based on characteristics of the scattered light.

In one embodiment, a system for determining the concentration of a component in a fluid sample may include a reaction vessel, a light source, a detection tube, and a mobile terminal. The reaction vessel may be configured to contain the fluid sample and a first reagent, and may be configured to receive a second reagent with a detection compound capable of interacting with the component in the fluid sample. The reaction vessel may include at least a first window. The light source may be configured to emit light into the reaction vessel, such that the detection compound and the component, when interacted and exposed to the light entering the reaction vessel, produces scattered light. The first window of the reaction vessel may be configured to allow the scattered light to exit the reaction vessel. The detection tube may include a first send and a second end, and may be configured to transmit the scattered light from the first end to the second end. The first end of the detection tube may be configured to engage the first window of the reaction vessel. The second end of the detection tube may be configured to reversibly engage a mobile terminal. The mobile terminal may be configured to determine the concentration of the component of interest based on characteristics of the scattered light.

In one embodiment, a system for determining the concentration of a component in a fluid sample may include a reaction vessel, a reagent vessel, a sealing cap, a light source, and a detection tube. The reaction vessel may be configured to contain the fluid sample and a first reagent, and include at least a first window. The reagent vessel may be configured to contain a second reagent with a detection compound capable of interacting with the component in the fluid sample, and may be configured to release the detection compound into the reaction vessel. The sealing cap may include a release feature and may be configured to reversibly seal the reaction vessel and the reagent vessel such that contents of the reaction vessel and the reagent vessel are separated by the sealing cap. Activation of the release feature may allow the detection compound within the reagent vessel to contact the component in the fluid sample. The light source may be configured to emit light into the reaction vessel, such that the detection compound and the component, when interacted and exposed to the light entering the reaction vessel, produces scattered light. The first window of the reaction vessel may be configured to allow the scattered light to exit the reaction vessel. The detection tube may include a first end and a second end, and may be configured to transmit the scattered light from the first end to the second end. The first end of the detection tube may be configured to engage the first window of the reaction vessel. The second end of the detection tube may be configured to reversibly engage a mobile terminal. The mobile terminal may be configured to determine the concentration of the component of interest based on characteristics of the scattered light.

Some embodiments disclosed herein relate to methods for determining the concentration of a component in a fluid sample. In one embodiment, a method for determining a concentration of a component in a fluid sample may include providing the system of as disclosed herein; providing a fluid sample with the component; contacting at least a portion of the fluid sample with a first reagent in the reaction vessel to create a sample media; providing a second reagent comprising a detection compound in a reagent vessel to create a detection media; sealing the reagent vessel and the reaction vessel with a sealing cap; activating the release feature to cause the detection media to contact the sample media in the reaction vessel, wherein the detection compound interacts with the component; activating the light source, wherein interaction of the detection compound with the component produces scattered light when exposed to light from the light source; detecting a rate of change of intensity of the scattered light with respect to time; determining a maximal rate of change of intensity of the scattered light, wherein the maximal rate of change of intensity of the scattered light is correlated to the concentration of the component within the fluid sample.

In one embodiment, a method for determining a concentration of a component in a fluid sample may include providing a fluid sample with the component; contacting at least a portion of the fluid sample with a first reagent to form a sample media; providing a second reagent that may include a detection compound to form a detection media; contacting the sample media with the detection media to form a reaction mixture, wherein the detection compound interacts with the component; exposing the reaction mixture to a light source, wherein interaction of the detection compound with the component produces scattered light when exposed to light from the light source; and determining the concentration of the component in the fluid sample based on characteristics of the scattered light using a mobile terminal.

In one embodiment, a method for determining a concentration of a component in a fluid sample may include providing a fluid sample with the component; contacting at least a portion of the fluid sample with a first reagent to create a sample media; providing a second reagent that may include a detection compound to create a detection media; contacting the sample media with the detection media to form a reaction mixture wherein the detection compound interacts with the component; exposing the reaction mixture to light from a light source, wherein interaction of the detection compound with the component produces scattered light when exposed to light from the light source; detecting a rate of change of intensity of the scattered light with respect to time; and determining a maximal rate of change of intensity of the scattered light, wherein the maximal rate of change of intensity of the scattered light is correlated to the concentration of the component within the fluid sample.

Some embodiments disclosed herein relate to methods of forming a system for determining a concentration of a component in a fluid sample. In one embodiment, a method of forming a system for determining a concentration of a component in a fluid sample includes providing a reaction vessel configured to contain the fluid sample, the reaction vessel comprising at least a first window, and the reaction vessel configured to receive a detection compound that interacts with the component in the fluid sample; providing a light source configured to emit light into the reaction vessel such that the detection compound and the component, when interacted and exposed to the light entering the reaction vessel, produces scattered light, and the scattered light exits the reaction vessel through the first window; providing a detection tube comprising a first end and a second end, the detection tube configured to transmit light from the first end to the second end, the first end of the detection tube configured to engage the first window of the reaction vessel to allow the scattered light that exits through the first window to pass from the first end to the second end of the detection tube, and the second end of the detection tube configured to reversibly engage a mobile terminal that is configured to detect the scattered light and to determine the concentration of the component based on characteristics of the scattered light.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 depicts one embodiment of a system disclosed herein for determining a concentration of a component in a fluid sample.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Nephelometry is widely used in clinical laboratories. However, investment costs in equipment and training of personnel can limit its wide applicability.

When used, nephelometry is employed to determine concentrations of targets of interest. For example, it is used in immunological analysis of patients to determine the concentrations of blood plasma proteins. Such a determination may be important in the clinical assessment of a patient after treatment, to monitor disease progression or relapse, and/or to make a diagnosis.

Nephelometry generally involves measurement of the turbidity of a liquid sample after light is passed through the sample. When a sample containing a target component of interest is combined with a detection compound that interacts with the target of interest, a suspension of small particles is generated. These particles are the result of the interaction of the detection compound with the target of interest. When light is passed through the sample, rather than absorbing all of the light, a certain amount of it will be deflected by the small particles and pass through the sample at an angle (for example, the light is scattered). By measuring the amount of light scattered by the particles, different properties of the sample can be measured and evaluated, such as the concentration of the target component of interest.

Nephelometry can be performed as end point nephelometry or kinetic nephelometry. Depending on the embodiment, either type may be used with the systems and methods disclosed herein. In some embodiments, end point nephelometry may be used when the amount of light scattered by the sample is measured after the detection compound and a fluid sample with the component of interest have had sufficient time to fully interact, so there is no change in the intensity of the scattered light. In some embodiments, kinetic nephelometry may be used when the rate of change of intensity of scattered light is measured after the detection compound is added to the fluid sample to interact with the component of interest. The rate of change of intensity of the scattered light is non-linear. The rate of change is typically slow at the inception of the reaction, but then increases to a peak rate before decreasing again (eventually to zero). The decrease in the rate can be due to depletion of either the component of interest or the detection compound. The peak value of the rate change of intensity of the scattered light can be correlated to the concentration of the component of interest. For example, the formation of an antigen-antibody complex may be measured based on change in the intensity of light scatter to determine the kinetics of the antigen-antibody binding speed in unit time, which is referred to as the “rate”. Under experimental conditions, the peak rate of binding is proportional to the content of the antigen, and corresponds to the peak intensity of scattered light. The amount of the antigen-antibody complex increases gradually with time, until the peak antigen-antibody binding rate is reached. The concentration of the component can then be obtained by data processing algorithms, such as, by comparison of the peak rate obtained to a known standard curve. Such methods can be repeated for various samples, and the differences in their respective concentrations of the target component of interest can be detected, and compared, if desired.

Consistent with embodiments disclosed herein, and discussed in more detail below, the systems and methods can be useful for the detection of a component of interest in a fluid sample. In several embodiments, the fluid sample is a non-biological fluid. In several embodiments, the fluid sample is an environmental sample, such as, for example, water from oceans, lakes, reservoirs, rivers, ponds, watersheds, aquifers, or other such site of water collection, either natural or man-made. In such embodiments, the component to be detected may be an environmental contaminant, such as, for example, a heavy metal, chemical wastes, sewage, and the like.

In several embodiments, the fluid sample is a biological fluid. In several embodiments, the biological liquid is blood, plasma, serum, urine, sputum, spinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid, or any combination thereof.

In some embodiments, fluid samples are optionally pre-treated. In several embodiments, where the fluid sample is whole blood, the fluid sample is pre-treated with a separating gel, a coagulating agent, or both.

In several embodiments, the component in the fluid sample is a protein, protein fragment, protein complex, peptide, small molecule, nucleic acid (or complex thereof), or other detectible entity. In several embodiments, the component is a IgA, IgG, IgM, IgD, Bence Jones protein, TP, Alb, a1-MG, a2-MG. B2-MG, Tf, CRP, a1-AG, AAG, a1-AT, HP, CER, Fbg, AT-III, A(SAA), RF, ASO, AdnaseB, CSF-IgG, CSF-IgA, CSF-IgM, ApoA-I, ApoA-II, ApoB, Apo-E, a(Lpa), IgE, PA, PLG, HGB, SF, 3(C3), 4(C4), 1q(C1q), Fn, CystatinC, hsCRP, T(TnT), MYO, HbAlc, or GA. In several embodiments, the component is an exogenous antigen, while in other embodiments, the component is an endogenous antigen. In still additional embodiments, the component is an autoantigen.

### Reagents

In several embodiments, systems for determining concentrations of components in fluid samples can be configured to work with one or more reagents. In several embodiments, the system may be configured to contact a first reagent with the fluid sample. In some embodiments the first reagent may include a diluent. In some embodiments, the first reagent may include a buffer solution, a coagulating agent, a surfactant, a stabilizer, a preservative, or any combination thereof.

In some embodiments, the buffer solution is a phosphate buffer solution. In several embodiments, the buffer solution comprises one or more buffering agents, such as citric acid, acetic acid, potassium dihydrogenphosphate (KH₂PO₄), N-Cyclohexyl-2-aminoethanesulfonic acid (CHES), and/or borate. Additional embodiments employ Mcllvaine's buffer solutions, Carmody buffer solutions and/or Britton-Robinson buffer solutions are used. In still additional embodiments, a buffer solution may comprise one or more of 3-{[tris(hydroxymethyl)methyl]amino}propanesulfonic acid (TAPS), N,N-bis(2-hydroxyethyl)glycine (Bicine), tris(hydroxymethyl)methylamine (Tris), N-tris(hydroxymethyl)methylglycine (Tricine), 3-[N-Tris(hydroxymethyl)methylamino]-2-hydroxypropanesulfonic Acid (TAPSO), 4-2-hydroxyethyl-1-piperazineethanesulfonic acid (HEPES), 2-{[tris(hydroxymethyl)methyl]amino}ethanesulfonic acid (TES); 3-(N-morpholino)propanesulfonic acid (MOPS), piperazine-N,N′-bis(2-ethanesulfonic acid) (PIPES), dimethylarsinic acid (Cacodylate), saline sodium citrate (SSC), 2-(N-morpholino)ethanesulfonic acid (MES), 2(R)-2-(methylamino)succinic acid (Succinic acid) or combinations of any of the foregoing.

In some embodiments, the coagulating agent is polyethylene glycol. In some embodiments, the coagulating agent is a polysorbate, such as polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), polysorbate 80 (polyoxyethylene (20) sorbitan monooleate), or combinations thereof.

In several embodiments, the surfactant may comprise sorbitan tristearate, sorbitan trioleate, sorbitan monooleate, sorbitan sesquioleate, sorbitan monopalmitate, or combinations thereof. In several embodiments, the surfactant may comprise a polysorbate, such as polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), polysorbate 80 (polyoxyethylene (20) sorbitan monooleate), or combinations thereof. In several embodiments, the surfactant is a hydrophilic polyethylene oxide chain, such as TRITON X-100 (C₁₄H₂₂O(C₂H₄O)ₙ, octylphenoxypolyethoxyethanol, polyether sulfonate, alkyl polyglucoside, or combinations thereof. Other nonionic surfactants may be used in some embodiments. Alternatively (or in combination), anionic surfactants can be used.

In several embodiments, the stabilizer comprises ethylenediaminetetraacetic acid (EDTA). However, other stabilizers may also be used. For example, iminodisuccinic acid, polyaspartic acid, Ethylenediamine-N,N′-disuccinic acid (EDDS), Methylglycinediacetic acid (MGDA), L-glutamic acid N,N-diacetic acid, tetra sodium salt (GLDA), or combinations thereof, are also used in several embodiments.

In several embodiments, the preservative comprises sodium azide or thimerosal. In several embodiments, biocidal compounds (e.g., PROCLIN 300, PROCLIN 950, PROCLIN B-119, etc.) or may also be used, either in place of or in addition to other preservatives.

In several embodiments, the pH of the reagents ranges from about 6.5 to about 8.5. In some embodiments, the pH of one or more of the reagents is about 6.5, about 6.7, about 6.9 about 7.0, about 7.1, about 7.2, about 7.3, about 7.4, about 7.5, about 7.6, about 7.7, about 7.8, about 7.9 about 8.1, about 8.3, about 8.5, or any pH value within those values listed.

In several embodiments, the system may be configured to contact the fluid sample and the first reagent with a second reagent. In some embodiments, the second reagent includes a detection compound. The detection compound may be capable of interacting with the component in the fluid sample.

In several embodiments, the detection compound is a molecule capable of interacting with the component of interest. In an embodiment, the detection compound is an antibody. For example, the antibody can be used to detect the presence of antigens, and the antigens detected by the antibody can be proteins. In an embodiment, the detection compound is an antiserum. In an embodiment, the detection compound is an antigen. In some embodiments, the second reagent further includes a buffer solution, such as phosphate buffer solution. In some embodiments, the component of interest may be an antibody, and in such cases, the detection compound would be an antigen. For example, in some embodiments, rheumatoid factor (RF) in a blood sample may be determined and in these embodiments, the component of interest would be antibody (RF) and the detection compound would be antigen (IgG).

In still additional embodiments, the detection compound may be a ligand or a receptor. For example, in several embodiments the detection compound may be a cytokine and the component of interest may be a soluble cytokine receptor. Alternatively, in several embodiments, the detection compound may be a probe or other small molecule that interacts with an antigen on a specific cell type in a fluid sample (for example, cancerous cells in a blood sample). In general, the detection compound can be any molecule that interacts with a component of interest in a manner that the resulting complex formed produces scattered light when exposed to light.

### Nephelometric Systems

In several embodiments, a system for determining the concentration of a component in a fluid sample is provided. In several embodiments, the system includes at least one reaction vessel, at least one light source, and at least one detection tube. In several embodiments, the system may also include at least one reagent vessel and at least one sealing cap. In several embodiments, the system may also include at least one mobile terminal.

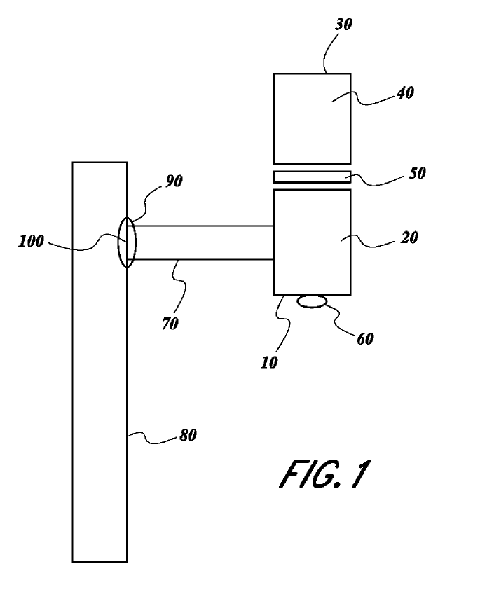

FIG. 1 depicts one non-limiting example system in accordance with the embodiments disclosed herein. A system for determining the concentration of a component in a fluid sample may include a reaction vessel 10 configured to contain a fluid sample and a first reagent 20. The first reagent 20 may be any of those described in the “Reagents” section above. The reaction vessel 10 may include at least a first window (not shown). The reaction vessel 10 may be configured to receive a second reagent 40 that include a detection compound capable of interacting with the component in the fluid sample. The second reagent 40 may be any of those described in the “Reagents” section above. A light source 60 may be configured to emit light into the reaction vessel 10, such that the detection compound and the component, when interacted and exposed to the light entering the reaction vessel, produces scattered light. The scattered light can exit the reaction vessel 10, for example, the first window of the reaction vessel 10 may be configured to allow the scattered light to exit the reaction vessel 10. A detection tube 70, having a first end and a second end, may be configured to transmit the scattered light from the first end to the second end. The first end of the detection tube may be configured to engage the reaction vessel 10. For example, the first end of the detection tube 70 may be configured to engage the first window of the reaction vessel 10. The second end of the detection tube 70 may be configured to reversibly engage a mobile terminal 80 at a second end. The mobile terminal 80 can be configured to determine the concentration of the component in the fluid sample based on characteristics of the scattered light. For example, a sealing ring 90 may be used to maintain a substantially sealed connection between the detection tube 70 and the mobile terminal 80. The mobile terminal 80 may include a sensor 100 configured to interact with the second end of the detection tube 70 such that the mobile terminal may be configured to determine the concentration of the component of interest based on characteristics of the scattered light. The light source 60 may be internal or external to the reaction vessel 10. In an embodiment, the light source 60 is positioned outside the reaction vessel 10, and the reaction vessel 10 may further include a second window which allows light from the light source 60 to be transmitted into the reaction vessel 10. The system may further include a reagent vessel 30 configured to contain the second reagent 40. As described herein, the second reagent may include the detection compound. Accordingly, the reagent vessel 30 may be configured to release the detection compound into the reaction vessel 10. For example, a release feature such as a sealing cap 50 may be configured to reversibly seal the reaction vessel 10 and the reagent vessel 30 such that the contents of both vessels are separated by the sealing cap 50. Activation of the release feature, such as by releasing the sealing cap 50, may allow the detection compound within the reagent vessel 30 to contact the component in the fluid sample. For example, when the sealing cap 50 is released, the detection compound may move from the reagent vessel 30 to the reaction vessel 10, or vice versa, and contact the component in the fluid sample.

### Vessels

As described herein, the system can include at least one reaction vessel. In several embodiments, the reaction vessel includes at least a first window. The first window, as described herein, may be configured to allow light from the reaction vessel (for example, scattered light) to exit the reaction vessel and through the detection tube to be detected by the mobile terminal to which the system is coupled. In some embodiments, the angle formed between the first window and the light source is greater than 0 degrees. In some embodiments, the angle formed between the first window and the light source is less than or equal to about 90 degrees. For example, in some embodiments, the angle between the window and the light source is about 0 degrees to about 10 degrees, about 10 degrees to about 20 degrees, about 20 degrees to about 30 degrees, about 30 degrees to about 40 degrees, about 40 degrees to about 50 degrees, about 50 degrees to about 60 degrees, about 60 degrees to about 70 degrees, about 70 degrees to about 80 degrees, about 80 degrees to about 90 degrees, or any value in between any two of these values (including endpoints).

In some embodiments, the reaction vessel further includes a second window. In some embodiments, the second window allows light to enter the reaction vessel from a light source positioned outside the reaction vessel. In some embodiments, the angle between the first window and the second window is greater than 0 degrees. In some embodiments, the angle between the first window and the second window is less than or equal to about 90 degrees. For example, in some embodiments, the angle between the window and the light source is in the range of about 0 degrees to about 10 degrees, about 10 degrees to about 20 degrees, about 20 degrees to about 30 degrees, about 30 degrees to about 40 degrees, about 40 degrees to about 50 degrees, about 50 degrees to about 60 degrees, about 60 degrees to about 70 degrees, about 70 degrees to about 80 degrees, about 80 degrees to about 90 degrees, or any value in between any two of these values (including endpoints).

In some embodiments, the reaction vessel can be configured to contain a fluid volume of about 10 μL to about 10 mL (for example, of the first reagent, the fluid sample or both). In some embodiments, the reaction vessel can be configured to contain about 10 μL to about 5 mL of the first reagent. For example, the reaction vessel can contain about 0 μL to about 10 μL, about 10 μL to about 20 μL, about 20 μL to about 30 μL, about 30 μL to about 40 μL, about 40 μL to about 50 μL, about 50 μL to about 60 μL, about 60 μL to about 70 μL, about 70 μL to about 80 μL, about 80 μL to about 90 μL, about 90 μL to about 100 μL, about 100 μL to about 200 μL, about 200 μL to about 300 μL, about 300 μL to about 400 μL, about 400 μL to about 500 μL, about 500 μL to about 600 μL, about 600 μL to about 700 μL, about 700 μL to about 800 μL, about 800 μL to about 900 μL, about 900 μL to about 1,000 μL, about 1 mL to about 2 mL, about 2 mL to about 3 mL, about 3 mL to about 4 mL, about 4 mL to about 5 mL, or any value in between these ranges (including endpoints), of the first reagent. In some embodiments, the reaction vessel can be configured to contain additionally about 10 μL to about 5 mL of the fluid sample. For example, the reaction vessel can contain about 0 μL to about 10 μL, about 10 μL to about 20 μL, about 20 μL to about 30 μL, about 30 μL to about 40 μL, about 40 μL to about 50 μL, about 50 μL to about 60 μL, about 60 μL to about 70 μL, about 70 μL to about 80 μL, about 80 μL to about 90 μL, about 90 μL to about 100 μL, about 100 μL to about 200 μL, about 200 μL to about 300 μL, about 300 μL to about 400 μL, about 400 μL to about 500 μL, about 500 μL to about 600 μL, about 600 μL to about 700 μL, about 700 μL to about 800 μL, about 800 μL to about 900 μL, about 900 μL to about 1,000 μL, about 1 mL to about 2 mL, about 2 mL to about 3 mL, about 3 mL to about 4 mL, about 4 mL to about 5 mL, or any value in between any two of these values (including endpoints) of the fluid sample.

As described herein, the system can also include at least one reagent vessel. In some embodiments, the reagent vessel may be configured to contain a second reagent that includes a detection compound capable of interacting with the component in the fluid sample. In some embodiments, the reagent vessel may be configured to contain about 10 μL to about 5 mL of the second reagent. For example, in some embodiments the reagent vessel can contain about 0 μL to about 10 μL, about 10 μL to about 20 μL, about 20 μL to about 30 μL, about 30 μL to about 40 μL, about 40 μL to about 50 μL, about 50 μL to about 60 μL, about 60 μL to about 70 μL, about 70 μL to about 80 μL, about 80 μL to about 90 μL, about 90 μL to about 100 μL, about 100 μL to about 200 μL, about 200 μL to about 300 μL, about 300 μL to about 400 μL, about 400 μL to about 500 μL, about 500 μL to about 600 μL, about 600 μL to about 700 μL, about 700 μL to about 800 μL, about 800 μL to about 900 μL, about 900 μL to about 1,000 μL, about 1 mL to about 2 mL, about 2 mL to about 3 mL, about 3 mL to about 4 mL, about 4 mL to about 5 mL, or any value in between any two of these values (including endpoints) of the second reagent.

In some embodiments, the reaction vessel may be configured to receive about 10 μL to about 5 mL of the second reagent, in addition to the volumes of the first reagent and the fluid sample discussed above. For example, in some embodiments the reaction vessel can receive about 0 μL to about 10 μL, about 10 μL to about 20 μL, about 20 μL to about 30 μL, about 30 μL to about 40 μL, about 40 μL to about 50 μL, about 50 μL to about 60 μL, about 60 μL to about 70 μL, about 70 μL to about 80 μL, about 80 μL to about 90 μL, about 90 μL to about 100 μL, about 100 μL to about 200 μL, about 200 μL to about 300 μL, about 300 μL to about 400 μL, about 400 μL to about 500 μL, about 500 μL to about 600 μL, about 600 μL to about 700 μL, about 700 μL to about 800 μL, about 800 μL to about 900 μL, about 900 μL to about 1,000 μL, about 1 mL to about 2 mL, about 2 mL to about 3 mL, about 3 mL to about 4 mL, about 4 mL to about 5 mL, or any value in between any two of these values (including endpoints) of the second reagent. In some embodiments, the reaction vessel can be configured to receive about 20 μL of the first reagent and about 20 μL of the second reagent, in addition to the fluid sample. Thus, in some embodiments, the reaction vessel may have a total volume of about 10 μL to about 15 mL of the fluid sample, the first reagent and the second reagent. For example, in some embodiments, the reaction vessel can be configured to receive a total volume of about 0 μL to about 10 μL, about 10 μL to about 20 μL, about 20 μL to about 30 μL, about 30 μL to about 40 μL, about 40 μL to about 50 μL, about 50 μL to about 60 μL, about 60 μL to about 70 μL, about 70 μL to about 80 μL, about 80 μL to about 90 μL, about 90 μL to about 100 μL, about 100 μL to about 200 μL, about 200 μL to about 300 μL, about 300 μL to about 400 μL, about 400 μL to about 500 μL, about 500 μL to about 600 μL, about 600 μL to about 700 μL, about 700 μL to about 800 μL, about 800 μL to about 900 μL, about 900 μL to about 1,000 μL, about 1 mL to about 2 mL, about 2 mL to about 3 mL, about 3 mL to about 4 mL, about 4 mL to about 5 mL, about 5 mL to about 6 mL, about 6 mL to about 7 mL, about 7 mL to about 8 mL, about 8 mL to about 9 mL, about 9 mL to about 10 mL, about 10 mL to about 11 mL, about 11 mL to about 12 mL, about 12 mL to about 13 mL, about 13 mL to about 14 mL, about 14 mL to about 15 mL, or any value in between any two of these values (including endpoints).

In some embodiments, the system includes a sealing cap that interacts with the reaction vessel to form a seal that is substantially impermeable to fluid and light. In some embodiments, the seal is liquid-tight and external light-tight. In some embodiments, the sealing cap is configured to be attached or unattached from the reaction vessel. In some embodiments, the sealing cap forms a seal that is substantially impermeable to fluid and light between the reaction vessel and the reagent vessel. In some embodiments, the sealing cap is configured to be reversibly attached or unattached from the reaction vessel and the reagent vessel. In some embodiments, the sealing cap has a snap fit to the reaction vessel, the reagent vessel, or both vessels. In other embodiments, the sealing cap has a threaded fit to the reaction vessel, the reagent vessel, or both vessels. In some embodiments, the sealing cap has a compression fit to the reaction vessel, the reagent vessel, or both vessels. In several embodiments, a variety of interactions may be used in the system (for example, a snap fit to the reaction vessel in conjunction with a threaded fit to the reagent vessel. In some embodiments, the sealing cap includes a release feature that allows the detection compound to move from the reagent vessel to the reaction vessel and contact the component in the fluid sample. In some embodiments, the release feature allows the components of the reagent vessel and reaction vessel to interact. In some embodiments, the release feature is a quick release feature activated by pressing (or releasing) a button on the sealing cap. In some embodiments, the release feature may be a knob, lever, slide switch, or other mechanism that enable the movement of the detection compound from the reagent vessel to the reaction vessel.

In some embodiments, the reaction vessel is made of glass, plastic, metal, or other suitable material. In some embodiments, the plastic material is polypropylene. In some embodiments, the reaction vessel material is opaque and does not allow the transmission of light external to the system into the reaction except through the second window. In some embodiments, the reaction vessel is transparent to light of a certain wavelength. In some embodiments, the reagent vessel is made of glass, plastic, metal, or another suitable material. In some embodiments, the plastic material is polypropylene. In some embodiments, the reagent vessel is transparent to light of a certain wavelength.

In some embodiments, at least one of the reaction vessel, the reagent vessel, the sealing cap, or any combination of them do not allow the transmission of light external to the system into the system, and light internal to the system is not transmitted outside the system.

### Light Sources

As described herein, the system includes a light source. In some embodiments, the light source is a laser. In some embodiments, the light source is an LED. In some embodiments, the light source is a silicon iodine bulb. In some embodiments, the system includes any stable light source with gratings or filters or any appropriate optical devices that may act as light sources.

In some embodiments, the light source emits light with a wavelength in the ultraviolet wavelength spectrum. In some embodiments, the wavelength of light emitted by the light source is in the range of about 250 nm to about 1,000 nm. In some embodiments, the wavelength of light emitted by the light source is in the range of about 340 nm to about 800 nm. In some embodiments, the wavelength of light emitted by the light source is in the range of about 400 nm to about 620 nm. For example, in some embodiments, the wavelength of light emitted from the light source is about 250 nm to about 300 nm, about 300 nm to about 340 nm, about 340 nm to about 350 nm, about 350 nm to about 360 nm, about 360 nm to about 370 nm, about 370 nm to about 380 nm, about 380 nm to about 390 nm, about 390 nm to about 400 nm, about 400 nm to about 410 nm, about 410 nm to about 420 nm, about 420 nm to about 430 nm, about 430 nm to about 440 nm, about 440 nm to about 450 nm, about 450 nm to about 460 nm, about 460 nm to about 470 nm, about 470 nm to about 480 nm, about 480 nm to about 490 nm, about 490 nm to about 500 nm, about 500 nm to about 510 nm, about 510 nm to about 520 nm, about 520 nm to about 530 nm, about 530 nm to about 540 nm, about 540 nm to about 550 nm, about 550 nm to about 560 nm, about 560 nm to about 570 nm, about 570 nm to about 580 nm, about 580 nm to about 590 nm, about 590 nm to about 600 nm, about 600 nm to about 610 nm, about 610 nm to about 620 nm, about 620 nm to about 630 nm, about 630 nm to about 640 nm, about 640 nm to about 650 nm, about 650 nm to about 660 nm, about 660 nm to about 670 nm, about 670 nm to about 680 nm, about 680 nm to about 690 nm, about 690 nm to about 700 nm, about 700 nm to about 710 nm, about 710 nm to about 720 nm, about 720 nm to about 730 nm, about 730 nm to about 740 nm, about 740 nm to about 750 nm, about 750 nm to about 760 nm, about 760 nm to about 770 nm, about 770 nm to about 780 nm, about 780 nm to about 790 nm, about 790 nm to about 800 nm, about 800 nm to about 850 nm, about 850 nm to about 900 nm, about 900 nm to about 950 nm, about 950 nm to about 1,000 nm, or any value in between these ranges (including endpoints). In some embodiments, the system includes a wavelength filter coupled to the light source and configured to filter light emitted from the light source toward the second window of the reaction vessel. The wavelength filter can, for example, provide a narrower range of wavelengths or a specific wavelength of light as required by the system.

In some embodiments, the system includes at least one collimating lens coupled to the light source, and configured to collimate light emitted from the light source toward the second window of the reaction vessel.

In some embodiments, the light source is powered from at least one external power supply, a mobile terminal, or both. In several embodiments, the light source is solar powered. In several embodiments, the light source operates from an AC power source or in other embodiments, a DC power source. In several embodiments, the light source is provided by a mobile device. In some embodiments, an application on a mobile device produces light that is used in the nephelometric measurements. This may include, for example, a flashlight or flashbulb generated by a mobile phone.

### Detection Tube

As described herein, the system can include at least one detection tube. In some embodiments, a first end of the detection tube is configured to engage the first window of the reaction vessel and a second end of the detection tube is configured to reversibly engage a mobile terminal.

In some embodiments, the detection tube is made of glass (for example, borosilicate glass, tempered glass, and the like), metal (for example, steel, stainless steel, cast iron, tool steel, alloy steel, aluminum, titanium, copper, magnesium, and the like), plastic or other polymer (for example, polystyrene, polyvinyl chloride, polyamide, polyethylene, polypropylene, combinations thereof, and the like).

In several embodiments, the detection tube functions to transmit scattered light from the reaction vessel to the mobile terminal. In some embodiments, the detection tube and reaction vessel do not allow the transmission of light external to the system into the system (for example, the reaction vessel, the detection tube, and so on), and light internal to the system is not transmitted outside the system. As a result, the system can be substantially or completely “light tight” so as to reduce inaccuracies in the detection of the target component. In some embodiments, the reaction vessel is transparent to light of a certain wavelength and the detection tube is not transparent to light of a certain. In some embodiments, the detection tube, reaction vessel, and reagent vessel do not allow the transmission of light external to the system (for example, the reaction vessel, the detection tube, the reagent vessel, and so on) into the system, and light internal to the system is not transmitted outside the system. In some embodiments, the reaction vessel and reagent vessel are transparent to light of a certain wavelength and the detection tube is not transparent to light of a certain wavelength. In some embodiments, the detection tube is optionally treated, for example with a reflective coating, to facilitate transmission of light within the tube from the first end of the tube to the second end of the tube.

In some embodiments, a region of engagement between the first end of the detection tube and the first window of the reagent vessel, and a region of engagement between the second end of the detection tube and the mobile terminal, are configured to substantially prevent light other than light from the light source into the system.

In some embodiments, the system includes at least one sealing ring. In some aspects, the sealing ring seals (optionally reversibly) a region of engagement between the second end of the detection tube and the mobile terminal so that the region substantially prevents light other than light from the light source into the system.

In some embodiments, the angle between the first end of the detection tube or the first window of the reaction vessel and the light source or light transmission path is greater than 0 degrees, or less than or equal to 90 degrees. For example, the angle formed between the window and the light source is in the range of about 0 degrees to about 10 degrees, about 10 degrees to about 20 degrees, about 20 degrees to about 30 degrees, about 30 degrees to about 40 degrees, about 40 degrees to about 50 degrees, about 50 degrees to about 60 degrees, about 60 degrees to about 70 degrees, about 70 degrees to about 80 degrees, about 80 degrees to about 90 degrees, or any value in between two of these values.

### Mobile Terminals

In several embodiments, the system can be coupled to the mobile terminal or may include at least one mobile terminal. In several embodiments, the mobile terminal may be a smartphone, a tablet, a laptop, a computer, a camera, a light detection device, or any combination of these.

FIG. 1 depicts a mobile phone 80 with a camera 100 attached to a second end of the detection tube 70. The non-limiting embodiment in FIG. 1 includes a sealing ring 90 to attach the second end of the detection tube 70 to the mobile phone 80. The first end of the detection tube attaches to the reaction vessel 10, which includes a light source 60 and sealing cap 50.

In some embodiments, the mobile terminal may power the light source. In some embodiments, the mobile terminal includes an optical sensor capable of detecting the amount of scattered light. In some embodiments, the mobile terminal may include gravity, direction, and acceleration sensors that may be used to calculate the mixing time for the first and second reagent and the reaction vessel's orientation. In several embodiments, the mobile terminal is equipped with one or more of a variety of sensors. For example, the mobile terminal may include a temperature sensor (for air and/or fluid temperatures), an accelerometer, a tilt sensor, a timer a humidity sensor, a sensor to detect ambient light, or any combination thereof. In several embodiments, the sensors are activated and provide data to the mobile terminal, which can integrate the information and provide a user of the device a protocol for using the device that is appropriate for the environmental conditions in which the device is used. For example, the mobile terminal may alter the time that a user should mix a detection compound with a fluid sample when a device is used in a warm or humid environment as compared to when it is used in a colder environment.

In some embodiments, characteristics of the scattered light such as light intensity and rate of change of light intensity may be detected by the mobile terminal. In some embodiments, the mobile terminal may be used to calculate the concentration of the component of interest in the fluid sample based on the characteristics (for example, light intensity and rate of change of light intensity, and the like) of the scattered light detected.

In some embodiments, the mobile terminal includes software that is configured to analyze the characteristics of scattered light to determine the concentration of the component in the fluid sample. In some embodiments, the software includes an application to determine the maximum rate of change of intensity of the scattered light. In some embodiments, the software includes an application to determine the concentration of the component of interest in the fluid sample based on the maximum rate of change of scattered light intensity. In some embodiments, the software determines the concentration of the component by comparing the maximum rate of change of intensity of the scattered light to a standard curve at the same reaction temperature.

In some embodiments, the mobile terminal can include at least one information database of standard curves for different reaction temperatures, components of interest, fluid sample types, or other variables. In some embodiments, the standard curves are provided by external methods, such as by scanning a 2-dimensional or 3-dimensional bar code. In some embodiments, the bar code is a quick-response (QR) code.

In some embodiments, the mobile terminal has the capability to scan a 2-dimensional or 3-dimensional bar code to calibrate the system for its current reaction conditions. In some embodiments, the bar code is a QR code. In some embodiments, detecting the rate of change of intensity of the scattered light may be performed by positioning the mobile terminal at an angle greater than 0 degrees, or less than or equal to 90 degrees from the light source. For example, in some embodiments, the angle formed between the mobile terminal and the light source is in the range of about 0 degrees to about 10 degrees, about 10 degrees to about 20 degrees, about 20 degrees to about 30 degrees, about 30 degrees to about 40 degrees, about 40 degrees to about 50 degrees, about 50 degrees to about 60 degrees, about 60 degrees to about 70 degrees, about 70 degrees to about 80 degrees, about 80 degrees to about 90 degrees, or any value in between any two of these values (including endpoints).

### Nephelometric Methods

Various methods for determining the concentration of a component in a fluid sample are provided herein, depending on the embodiment. In several embodiments, the method includes: providing the fluid sample that includes the component, contacting at least a portion of the fluid sample with a first reagent to form a sample media; providing a second reagent that includes a detection compound to form a detection media; contacting the sample media with the detection media to form a reaction mixture, wherein the detection compound interacts with the component; exposing the reaction mixture to a light source, wherein interaction of the detection compound with the component produces scattered light when exposed to light from the light source; and determining the concentration of the component in the fluid sample based on characteristics of the scattered light using the mobile terminal. The determining of the concentration can be achieved by detecting a rate of change of intensity of the scattered light intensity with respect to time, for example after passing light through the reaction mixture; and determining a maximal rate of change of intensity of the scattered light. As the maximal rate of change of intensity of the scattered light can be correlated to concentrations of particular substances in fluid samples, it is possible to determine the concentration of the component in the fluid sample.

In one embodiment, a reaction vessel is configured to receive the first reagent. A fluid sample to be tested may be pre-treated with one or more pre-treatment techniques and then added to the reaction vessel with the first reagent. Alternatively, the first reagent may be pre-treated with one or more pre-treatment techniques and added to the reaction vessel with the fluid sample. A reagent vessel can be configured to receive a second reagent that includes a detection compound. The reaction vessel and the reagent vessel can then covered by a sealing cap, and the cap can be opened (for example, activated or released) to allow the contents of the reaction vessel and reagent vessel to contact one another. Mixing, agitation, shaking, or a combination thereof can be optionally performed to facilitate interaction between the component and the detection compound. As described herein, the interaction between the detection compound and the component produces scattered light when exposed to light from the light source. Accordingly, a light source can be activated and the rate of change of intensity of the scattered light with respect to time during the course of the interaction may be detected by the mobile terminal. In some cases, the mixing time and the vessel's orientation can be determined by gravity, direction, and acceleration sensors within the mobile terminal. In some embodiments, the sensors are able to evaluate the speed and orientation at which the reaction vessel is mixed, shaken, or flipped so that the mobile terminal may adjust the total mixing time necessary for the reaction. As discussed herein, for example, the mobile terminal may alter the time that a user should mix a detection compound with a fluid sample when the fluid sample is cold versus a fluid sample at a physiological temperature. Advantageously, the gravity sensor, direction and acceleration sensors, etc. of the mobile terminal ensure that the reagents are fully and appropriately mixed to result in a complete reaction and accurate results. Moreover, because the standing curves are embedded in the software of the mobile terminal, recalibration with new batches of reagents is unnecessary (though it can optionally be performed).

After determining the rate of change of intensity of the scattered light with respect to time, a final determination of the concentration of the component may be calculated. In some embodiments, the concentration of the component in the fluid sample may be calculated by comparing the maximum rate of change of intensity of the scattered light to a standard curve for a given compound or complex. In some embodiments, the result of this comparison can be adjusted based on external temperature, humidity, altitude, or other environmental factors that may appear to alter the concentration of the component as compared to a standard curve obtained under controlled laboratory conditions.

In some embodiments, detecting the rate of change of intensity of the scattered light can be measured for a period of time such that a maximal rate of change of intensity of the scattered light is detected and thereafter a decrease in the rate of change of intensity of the detected scattered light occurs. In some embodiments, detecting the rate may occur for about 30 seconds to about 10 minutes. In some embodiments, the amount of time during which the detection is performed may be about 30 seconds to about 40 seconds, about 40 seconds to about 50 seconds, about 50 seconds to about 60 seconds, about 1 minute to about 2 minutes, about 2 minutes to about 3 minutes, about 3 minutes to about 4 minutes, about 4 minutes to about 5 minutes, about 5 minutes to about 6 minutes, about 6 minutes to about 7 minutes, about 7 minutes to about 8 minutes, about 8 minutes to about 9 minutes, about 9 minutes to about 10 minutes, or shorter or longer time periods, depending on the reaction and/or environmental conditions.

In some embodiments, contacting the sample media with the detection media to form a reaction mixture may be performed for a period of time such that the detection compound and the component can interact. In some embodiments, the mixing may occur for about 30 seconds to about 10 minutes. For example, the mixing can occur for about 30 seconds to about 40 seconds, about 40 seconds to about 50 seconds, about 50 seconds to about 60 seconds, about 1 minute to about 2 minutes, about 2 minutes to about 3 minutes, about 3 minutes to about 4 minutes, about 4 minutes to about 5 minutes, about 5 minutes to about 6 minutes, about 6 minutes to about 7 minutes, about 7 minutes to about 8 minutes, about 8 minutes to about 9 minutes, about 9 minutes to about 10 minutes, or shorter or longer time periods, depending on the reaction and/or environmental conditions.

Some embodiments disclosed herein provide a method of determining the concentration of the component in a fluid sample using the systems described herein. In several embodiments, the method includes providing a fluid sample that contains a component of interest at a certain, but unknown, concentration. A user loads at least a portion of the fluid sample in to a reaction vessel, and further adds a volume of a first reagent to the reaction vessel, thereby creating a sample media. A second reagent that contains a detection compound is added to a reagent vessel to create a detection media. The reagent vessel and the reaction vessel may be sealed with a sealing cap that is between the two vessels. Based on the environmental temperature, humidity, temperature of the fluid sample, and the like, that are sensed by a mobile terminal, a protocol may be generated for the user to follow. The user then activates a release feature of the sealing cap that causes (or allows) the detection media to contact the sample media in the reaction vessel. The user mixes the two media for a period of time, as directed by the mobile device. In several embodiments, the mobile device can prompt a user to extend the mixing time, reduce the mixing time, increase the intensity of mixing, and so on based on the sensors of the mobile device. The user then places a light source (in some embodiments, provided as part of the mobile terminal) in communication with a first window of the reaction vessel and activates the light source. Within the reaction vessel, the interaction of the detection compound with the component produces a complex that when exposed to light from the light source causes a scattering (for example, a deflection) of the light. The scattered light passes out of the reaction vessel via a second window and through the detection tube. The mobile terminal senses the characteristics of the scattered light. For example, the mobile terminal detects the rate of change of intensity of the scattered light with respect to time, and determines the maximal rate of change of the intensity of the scattered light. The maximal rate of change of scattered light intensity may be correlated to the concentration of the component within the fluid sample, such as by comparison to a standard curve of data points.

Some embodiments disclosed herein provide a method of forming a system for determining a concentration of a component in a fluid sample. In several embodiments, the method includes providing a reaction vessel with at least a first window. The reaction vessel may be configured to contain the fluid sample, and may be configured to receive a detection compound capable of interacting with the component in the fluid sample. A light source may be provided and may be configured to emit light into the reaction vessel such that the detection compound and the component, when interacted and exposed to the light entering the reaction vessel, produces scattered light, and the scattered light exits the reaction vessel through the first window. A detection tube having a first end and a second end may be provided such that the first end of the detection tube is configured to engage the first window of the reaction vessel, to allow the scattered light that exits through the first window to pass from the first end to the second end of the detection tube, and the second end of the detection tube is configured to reversibly engage the mobile terminal. The mobile terminal can be configured to detect the scattered light and to determine the concentration of the component based on the characteristics of the scattered light. In some embodiments, the method of forming the system may further include generating a software application for installation on the mobile terminal. The software application may be configured to receive a signal from the mobile terminal corresponding to an amount of scattered light detected by the mobile device, and to determine a maximal rate of change of intensity of the scattered light which can be correlated to the concentration of the component in the fluid sample. The software application may also generate an output to indicate to a user of the system the concentration of the component in the fluid sample.

### Examples

### Example 1

### Detection of C-reactive Protein (CRP) in Blood Samples

A system to determine the concentration of C-reactive protein in a blood sample is provided. Appropriate application software is equipped on a mobile terminal by using the device to scan two-dimensional codes (to identify the reagents to be used, component to be detected, and sample type). The system includes a reaction vessel, a reagent vessel, a sealing cap, a light source, and a detection tube. The reaction vessel contains the fluid sample and a first reagent. The reaction vessel includes a first window. The reagent vessel contains a second reagent with a detection compound, and is configured to release the detection compound into the reaction vessel. The sealing cap includes a release feature that is configured to reversibly seal the reaction vessel and the reagent vessel such that the contents of the reaction vessel and reagent vessel are separated by the sealing cap. Activation of the release feature allows the detection compound within the reagent vessel to move from the reagent vessel to the reaction vessel and contact the component in the fluid sample. The release feature includes a sliding member that when slid from a first position to a second position, generates an opening between the reaction vessel and the reagent vessel. The light source, which is configured to emit light into the reaction vessel, is activated, causing the detection compound and the component of interest to interact and to scatter light. The scattered light exits the reaction vessel through the first window of the reaction vessel and through the detection tube. The detection tube is configured to engage the first window of the reaction vessel at a first end and to engage a mobile terminal at a second end such that the mobile terminal may receive the scattered light. The mobile terminal can then calculate the concentration of the component of interest based rate of change of the intensity of the scattered light, which is related to the concentration of the component of interest.

The system is connected to a light source (e.g., 600 nm LED light source) and 20 μL of blood from a fingertip is taken and transferred to a sample dilution tube with a dosing pipette. The blood sample is pre-treated by mixing evenly with 180 μl of a diluent, which includes buffer solution (0.02M phosphate buffer at pH 7.4) and stabilizer (EDTA). Next, 20 μL of the pretreated blood sample is added to a reaction vessel that contains 200 μl of the first reagent, which includes phosphate buffer (0.2M, at pH7.4), polyethylene glycol (PEG 6000), and preservative (sodium azide), and placed in the reaction vessel of the system. The total mixture is mixed by reversing the reaction vessel upside down according to instruction prompts from the mobile terminal software. A detecting system in the mobile terminal detects the signal strength and temperature of the reaction. After the sample, diluent and first reagent are mixed for two minutes, the mobile terminal prompts a user to add the second reagent (50 μl) from the reagent vessel, which included phosphate buffer (0.2M at pH 7.4) and c-reactive protein antibody, as well as latex microspheres. The sealing cap is broken between the reaction vessel and reagent vessel by pressing the release button on the reagent vessel lid so that the first and second reagents could mix. The reaction vessel is reversed upside down once and then placed on a shelf (for 30 seconds at room temperature) until the light source was activated and a mobile terminal generates a test report for the concentration of C-reactive protein from the sample based on the characteristics of the scattered light.

### Example 2

### Detection of Albumin in Urine Samples

Advantageously, the systems disclosed herein can also be used with other sample types. For example, the system as discussed above can also be used to detect albumin in a urine sample in order to detect albuminuria).

The system is connected to a light source (e.g., 600 nm LED light source) and 20 μL of urine is taken and transferred to a sample dilution tube with a dosing pipette. The urine sample urine sample is added to a reaction vessel that contains 200 μl of the first reagent, which includes phosphate buffer (0.2M, at pH7.4), polyethylene glycol (PEG 6000), and preservative (sodium azide), and placed in the reaction vessel of the system. The total mixture is mixed by reversing the reaction vessel upside down according to instruction prompts from the mobile terminal software. A detecting system in the mobile terminal detects the signal strength and temperature of the reaction. After the first reagent is mixed for two minutes, the mobile terminal prompts a user add the 50 μl of the second reagent from the reagent vessel, which includes phosphate buffer (0.2M at pH 7.4) and albumin antibody, and sodium azide. The sealing cap is broken between the reaction vessel and reagent vessel by pressing the release button on the reagent vessel lid so that the first and second reagents could mix. The reaction vessel is reversed upside down once and then placed on a shelf (for 30 seconds at room temperature) until the light source was activated and a mobile terminal generates a test report for the concentration of albumin from the sample based on the characteristics of the scattered light.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A system for determining a concentration of a component in a fluid sample, the system comprising:
a reaction vessel configured to contain the fluid sample and a first reagent, the reaction vessel comprising at least a first window; wherein the reaction vessel is configured to receive a second reagent comprising a detection compound capable of interacting with the component in the fluid sample; and
a light source configured to emit light into the reaction vessel; wherein the detection compound and the component, when interacted and exposed to the light entering the reaction vessel, produces scattered light; and
wherein the first window of the reaction vessel is configured to allow the scattered light to exit the reaction vessel;
wherein the first window of the reaction vessel is configured to reversibly engage a mobile terminal that detects the scattered light exiting the reaction vessel; and
wherein the mobile terminal is configured to determine the concentration of the component based on characteristics of the scattered light.

2. The system of claim 1, further comprising:
a detection tube comprising a first end and a second end, and configured to transmit the scattered light from the first end to the second end; wherein the first end of the detection tube is configured to engage the first window of the reaction vessel; and wherein the second end of the detection tube is configured to reversibly engage the mobile terminal.

3. The system of claim 1, wherein the light source is positioned outside the reaction vessel, and wherein the reaction vessel further comprises a second window, the second window allowing light from the light source to be transmitted into the reaction vessel.

4. The system of claim 1, further comprising a reagent vessel configured to contain the second reagent.

5. The system of claim 1, further comprising a sealing cap configured to interact with the reaction vessel and to form a seal with the reaction vessel, wherein the seal is substantially impermeable to fluid and light.

6. The system of claim 5, wherein the sealing cap is configured to reversibly interact with the reaction vessel.

7.-12. (canceled)

13. The system of claim 3, further comprising a collimating lens coupled to the light source, wherein the collimating lens is configured to collimate light emitted from the light source toward the second window of the reaction vessel.

14. The system of claim 3, further comprising a wavelength filter coupled to the light source, wherein the wavelength filter is configured to filter light emitted from the light source toward the second window of the reaction vessel.

15. The system of claim 1, wherein the first reagent comprises a buffer solution, a coagulating agent, a surfactant, a stabilizer, a preservative, or any combination thereof.

16.-19. (canceled)

20. The system of claim 1, wherein the second reagent further comprises a buffer solution.

21.-23. (canceled)

24. The system of claim 1, wherein the reaction vessel and the detection tube do not allow transmission of light external to the system into the reaction vessel or the detection tube, and light internal the reaction vessel or the detection tube is not transmitted outside the system.

25. (canceled)

26. The system of claim 1, wherein a region of engagement between the first end of the detection tube and the first window of the reaction vessel, and a region of engagement between the second end of the detection tube and the mobile terminal are configured to substantially prevent light other than light from the light source into the system.

27. (canceled)

28. The system of claim 1, wherein the mobile terminal comprises software configured to analyze the characteristics of the scattered light to determine the concentration of the component in the fluid sample.

29.-31. (canceled)

32. The system of claim 1, wherein the fluid sample is a non-biological fluid.

33. (canceled)

34. The system of claim 1, wherein the fluid sample is one or more selected from the group consisting of blood, plasma, serum, urine, sputum, spinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid, and any combination thereof.

35. The system of claim 1, wherein the component is a protein.

36. The system of claim 1, wherein the detection compound is one or more selected from the group consisting of an antibody, an antiserum, an antigen, and a combination thereof.

37.-40. (canceled)

41. The system of claim 1, wherein the light emitted from the light source has a wavelength of about 250 nm to about 1000 nm.

42.-45. (canceled)

46. The system of claim 1, wherein an angle formed between the first window and the light source is less than or equal to 90 degrees.

47.-70. (canceled)

71. A method for determining a concentration of a component in a fluid sample, the method comprising:
providing the fluid sample comprising the component;
contacting at least a portion of the fluid sample with a first reagent to create a sample media;
providing a second reagent comprising a detection compound to create a detection media;
contacting the sample media with the detection media to form a reaction mixture, wherein the detection compound interacts with the component;
exposing the reaction mixture to light from a light source, wherein interaction of the detection compound with the component produces scattered light when exposed to light from the light source;
detecting a rate of change of intensity of the scattered light with respect to time; and
determining a maximal rate of change of intensity of the scattered light, wherein the maximal rate of change of intensity of the scattered light is correlated to the concentration of the component within the fluid sample.

72.-73. (canceled)
